(19) United States
(12) Patent Application Publication
(10) Pub. No.: US 2005/0233467 A1
(43) Pub. Date: Oct. 20, 2005

(54) Visible wavelength fluorescent calcium indicators that are (i) leakage resistant and (ii) operate near membranes

(76) Inventors: Akwasi Minta, Austin, TX (US); Rogelio Escamilla, Kyle, TX (US); Earl Allen Bates, Kyle, TX (US)

Correspondence Address: RICK B. YEAGER, ATTORNEY, 10805 MELLOW LANE, AUSTIN, TX, 78759, US

(21) Appl. No.: 10/824,786

(22) Filed: Apr. 15, 2004

(51) Int. Cl. G01N 33/20

(52) U.S. Cl. 436/166; 436/79

(57) ABSTRACT

A new class of visible wavelength fluorescent calcium indicators with the BAPTA-like portion rendered zwitterionic by introduction of amine and carboxylic groups. “Fluo” compounds generally function with no extra ionized groups. The modified BAPTA moiety confers new properties while retaining ion selectivity and pH insensitivity. The dyes demonstrate reduced cell leakage and improved ability to study calcium near the cell membrane and preserve the fluorescent properties of “fluo” dyes. The modifications include (a) piperazinoacetic acid XIII for the leakage resistance (FLUO-LR), (b) dodecylpiperazine XIII for a near membrane indicator (FLUO-MOMO, FLUO-NOMO amphipathic “fluo” indicators which bind to cellular membranes and respond to calcium near the membrane), and (c) a propionic acid XII for general leakage resistance -FLUO-LOJO, FLUO-KOJO “fluo” indicators with an extra charge that enables them to resist leakage).

### FIELD OF THE INVENTION

The present invention relates to new visible wavelength calcium indicators which (1) do not leak out of cells or (2) bind to the plasma membrane and report calcium levels near the membrane. The new indicators include (1) FLUO-LOJO and FLUO-KOJO, “fluo” indicators with an extra charge that enables them to resist leakage and compartmentalization, (2) FLUO-NOMO and FLUO-MOMO—amphipathic “fluo” indicators which bind to cellular membranes and respond to calcium near the membrane. As “fluo” compounds, they retain the fluorescence attributed to “fluo” compounds, but they have a modified BAPTA moiety that confers these new properties on them while retaining their ion selectivity and pH insensitivity.

### BACKGROUND OF THE INVENTION

Changes in intracellular free calcium in the activities of the cell are responsible for many physiological functions, including neurosynaptic transmission, secretion of hormones, muscle contraction, cell division, memory, etc. A proper understanding of cell physiology therefore depends upon the ability to monitor changes in intracellular calcium concentration. The introduction of the first calcium indicators, BAPTA and Quin-2, by Dr. Tsien¹ in 1980, started a revolutionary new approach for studying calcium in individual and populations of cells. Since then, there have been more indicators that are brighter and have unique properties. For example, Fura-2 and Indo- 1² were the first dyes to be developed that made it possible to measure calcium based on the ratio of fluorescence intensity at two excitation or emission wavelengths. The feasibility of ratio-based measurements spawned the development of fluorescence-ratio fluorometric instruments and imaging systems, and has led to a multitude of new findings on the spatial and temporal aspects of calcium regulation in cells. The advancement in the technology also revealed several problems related to dye loading, such as leakage or compartmentalization, spectral alterations between intracellular dye and that in free solution, and unwanted binding to cellular constituents. These problems have revealed the apparent inaccuracies in how the dyes report the changes in calcium. One particular problem that was observed throughout studies with Fura-2 was that of leakage and compartmentalization. Dr. Poenie (in 1984) noted a consistent failure of Fura-2 to load in PTKI cells and tried to improve loading using Pluronic F127, and saw rapid leakage into perinuclear vesicles. The leakage was retarded by incubating cells at lower temperatures—e.g. 25° instead of 37°. The same phenomenon was observed by Hepler and Poenie in Trascendentia cells. The one indicator that seemed to stay in the cell was Rhod-2, which has a positive charge, but the positive charge made loading (dye going across the hydrophobic membrane) difficult and had a tendency to go into the mitochondria. Also, Rhod-2 does not have the unique property of ratiometry that Fura-2 has, and the increase in fluorescence upon binding calcium is not as large as that of other non-ratiometric dyes such as FLUO-3.

The design of leakage resistant Fura-2, now known as Fura-PE3 (Poenie & Minta), took into account the possibility of generating a zwitterion inside the cell. Thus, a tertiary amine with a side arm containing carboxylic acid which led to a protonation inside the cell was designed and properly executed to give a leakage resistant Fura-2³. A tertiary amine with a long hydrophobic alkyl chain, FFP18³, also gave a dye that stayed in the cell and reported calcium near the membrane. Fura-2, however, is an indicator which is excitable in ultraviolet light and has found great application in the study of calcium, because measurements of calcium are made based upon the ratio of fluorescent intensity at two excitation wavelengths, 380 nm/340 nm. The ratiometric measurement eliminates variables, such as degree of cell loading, photobleaching, detector sensitivity, cell thickness, optical thickness, and indicator concentration. The high energy of the UV light subjects cells to photodynamic injury, and the shorter wavelengths demand expensive quartz optics and cause interference in fluorescence from nucleic acids and aromatic amino acids.

Currently, the most popular dye for studying calcium is called “Fluo”, and there are two variations; FLUO-3^{4,6} and FLUO-4^{5,6}. These “fluo-” dyes are absorbed by visible light, but do not shift wavelength upon binding calcium. In this invention, we have applied the principle of generating leakage resistant and membrane fura-dyes by developing a synthesis that maintains the optical properties and provides these new properties as well. The near membrane “fluo” behaves in a similar way to the UV analog, but the leakage resistant version requires a modification to give the same result. This invention, therefore, provides visible wavelength analogs, thus overcoming the problems due to autofluorescence, and possible photodynamic injury due to ultraviolet light. The “fluo” compounds exhibit a large increase in fluorescence; therefore small changes in calcium concentration can be measured, compensating for the lack of ratiometry.

### SUMMARY OF THE INVENTION

The present invention is a new family of calcium indicators based on the “fluo” fluorophore, but modified to make them leakage resistant and to target the membrane instead of the cytosol. These new indicators are derived from a modified BAPTA backbone which contains a flexible attachment point for adding new functionality to the indicator without significantly affecting chelation or the fluorescent characteristics of the chromophore. There are three compounds, each with a different function from a regular “fluo” indicator. The first derivative, FLUO-MOMO or FLUO-NOMO, contains a hydrophobic tail which targets the indicator to cell membranes. This reports calcium transient in the membrane, whereas all regular “fluo” compounds target the cytosol. The second, FLUO-LR, targets the mitochondria, presumably because of a positively charged nitrogen in the piperazine moiety; regular “fluo” does not discriminate between mitochondria, cytosol and other organelles. Unlike regular “fluo” compounds, this new dye does not respond to calcium in the cytosol. The third compound, FLUO-LOJO or FLUO-KOJO, has an extra negative charge that forms salts in the cytosol that help it to stay without leaking out.

It is an object of this invention to provide a new class of visible wavelength fluorescent calcium indicators with specific targets in the cellular environment.

It is a further object of the present invention to provide visible wavelength fluorescent calcium indicators that do not leak out of the cell like the existing visible wavelength fluorescent calcium indicators.

It is a further object of the present invention to provide visible wavelength fluorescent calcium indicators that operate near membranes.

It is a further object of this invention to produce visible wavelength fluorescent calcium indicators that can be converted to acetoxymethyl esters so that they can be loaded into cells conventionally.

### BRIEF DESCRIPTION OF THE DRAWINGS

These and other objects and advantages of the present invention are set forth below and further made clear by reference to the drawings, wherein:

FIG. 1—Synthesis of main precursor.

FIG. 2—Synthesis from precursor to the three dyes.

FIG. 3—Alternate synthesis for FLUO-LOJO & FLUO-KOJO.

FIG. 4—Calcium titration of FLUO-LOJO.

FIG. 5—Calcium titration of FLUO-KOJO.

FIG. 6—Comparison of leakage of FLUO-4(AM) with FLUO-LOJO(AM), courtesy of Dr. Aiden Hampson of Cortex Pharmaceuticals.

FIG. 7—Localization of FLUO-MOMO near the membrane and its response to calcium, courtesy of Dr. Blaustein of The University of Maryland Medical School.

FIG. 8—Localization of FLUO-LR in the mitochondria, courtesy of Dr. Raul Martinez-Zaguillan of Texas Tech University.

### DEFINITION OF TERMS USED HEREIN

In the present specifications and claims, reference will be made to phrases and terms of art which are expressly defined for use herein as follows:

As used herein [Ca²⁺] means intracellular free calcium.

As used herein EGTA means ethylene glycol bis (-beta-aminoethylether)-N,N,N′,N′-tetraacetic acid.

As used herein BAPTA means 1,2-bis(aminophenoxy)ethane N,N,N′,N′-tetraacetic acid; the chemical structure for BAPTA is:

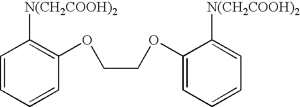

As used herein “BAPTA-like” means substituted derivatives of BAPTA, which retain the essential characteristics of two bis (carboxymethyl) amino-substituted phenyl rings, said rings being linked at the positions ortho to the amines through a four atom bridge, wherein the atom adjacent to the phenyl ring is oxygen and the two center arms are each carbon.

As used herein FLUO-3 means 9-(4-biscarboxymethyl)amino-3-(2-(2-biscarboxymethyl)amino-5 -methylphenoxy)ethoxy)phenyl)2,7-dichloro-6-hydroxy-3H-xanthen-3-one.

As used herein FLUO-4 means 9-(4-bis(carboxymethyl)amino-3-(2-(2-bis(carboxymethyl)amino-5-methylphenoxy)ethoxy)phenyl)-2,7 difluoro-6-hydroxy-3H-xanthen-3-one. FIG.

As used herein FLUO-LOJO is a derivative of FLUO-4. (FIG. XII) X=F

As used herein FLUO-KOJO is a derivative of FLUO-3. (FIG. XII) X=Cl

As used herein FLUO-MOMO is a hydrophobic derivative of FLUO-4. (FIG. XIII) X=F

As used herein FLUO-NOMO is a hydrophobic derivative of FLUO-3. (FIG. XIII) X=Cl

As used herein mM means 10⁻⁶ moles/liter and mM means 10⁻⁹ moles/liter.

As used herein AM¹² ester means AcetoxyMethyl ester.

### DETAILED DESCRIPTION OF THE DRAWINGS

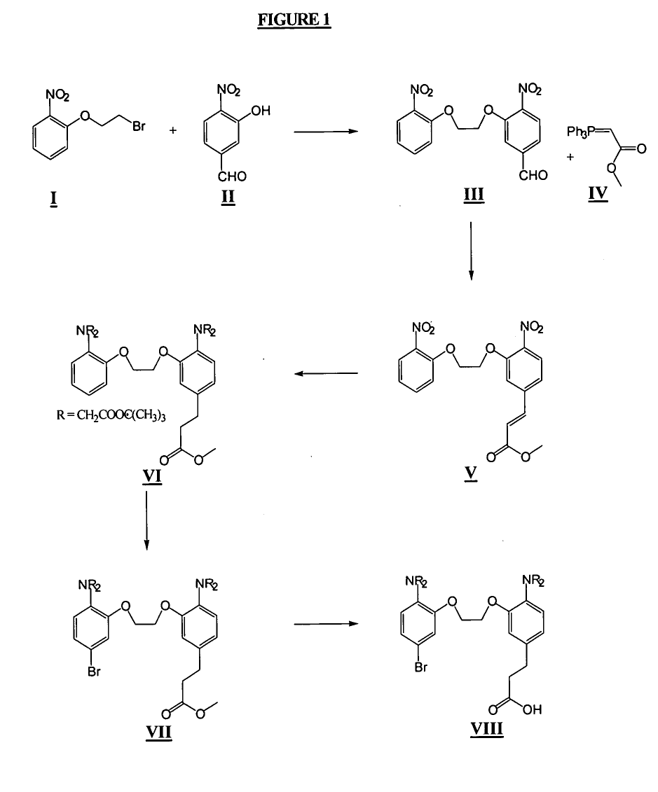

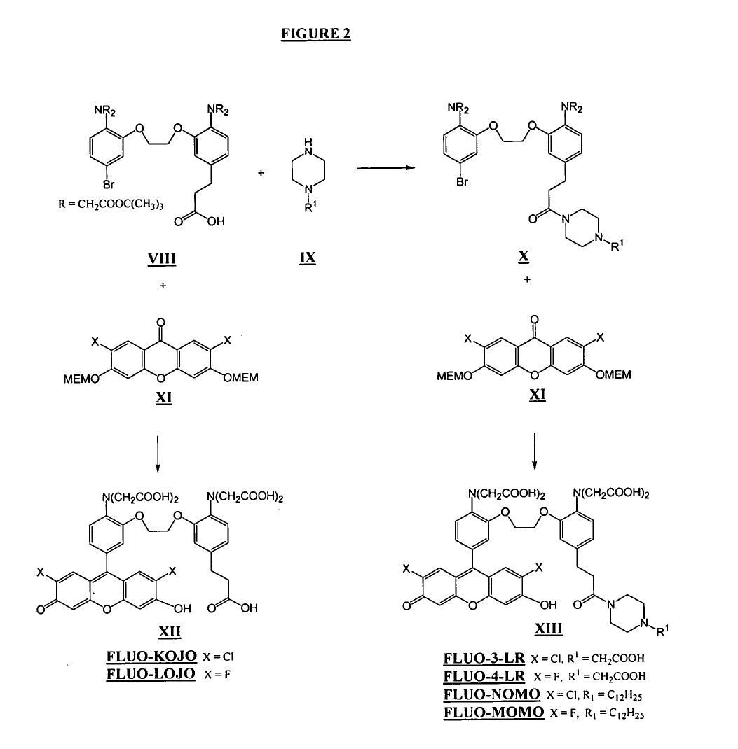

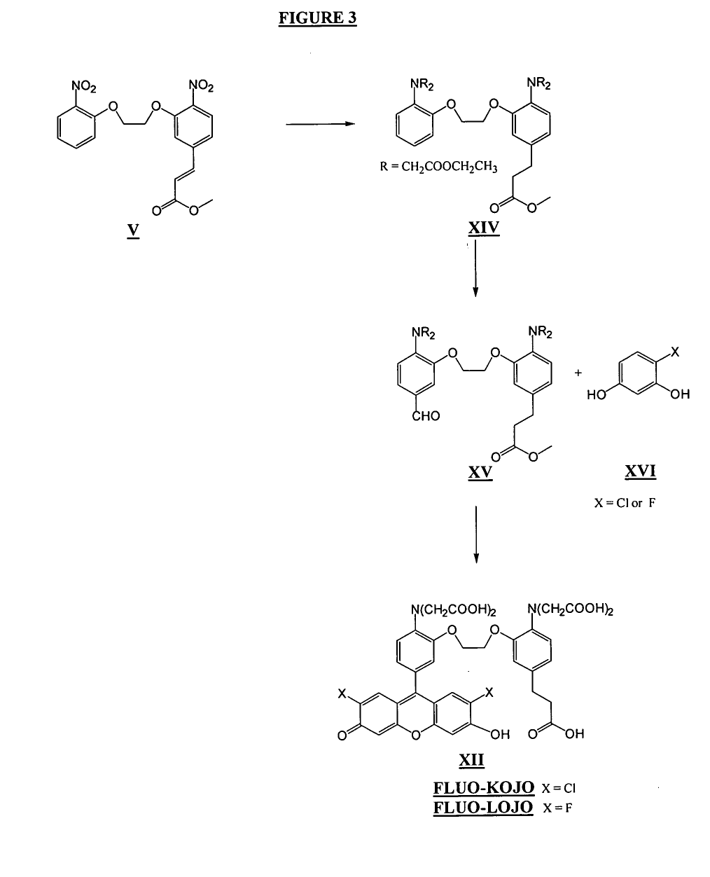

FIGS. 1 and 2 show the synthetic pathway leading to all three compounds. FIG. 1 shows how the key intermediate (VII) was made and FIG. 2 shows the conversion to all three compounds. FIG. 3 is an alternate synthesis to FLUO-LOJO and FLUO-KOJO. The Roman numerals used in the figure are keyed to the synthetic details discussed in the Methods of Synthesis section.

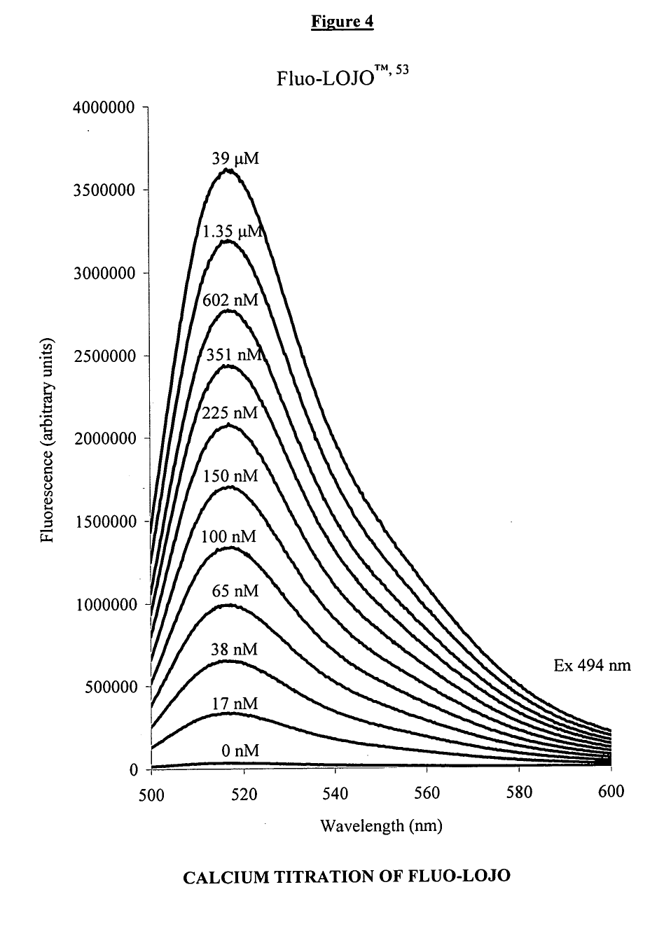

FIG. 4 shows a titration of FLUO-LOJO with calcium. It is an emission spectra of Ca²⁺ titration of FLUO-LOJO in 10 mM EGTA, 100 mM KCl, 30 mM MOPS buffer at pH 7.2. Excitation was set at 494 nm. 0 nM, 17 nM, 38 nM, 65 nM, 100 nM, 150 nM, 225 nM, 351 nM, 602 nM, 1.35 μM, and 39 μM of Ca²⁺ were added in sequence. The temperature was 25° and that gave a dissociation constant of 400 nM.

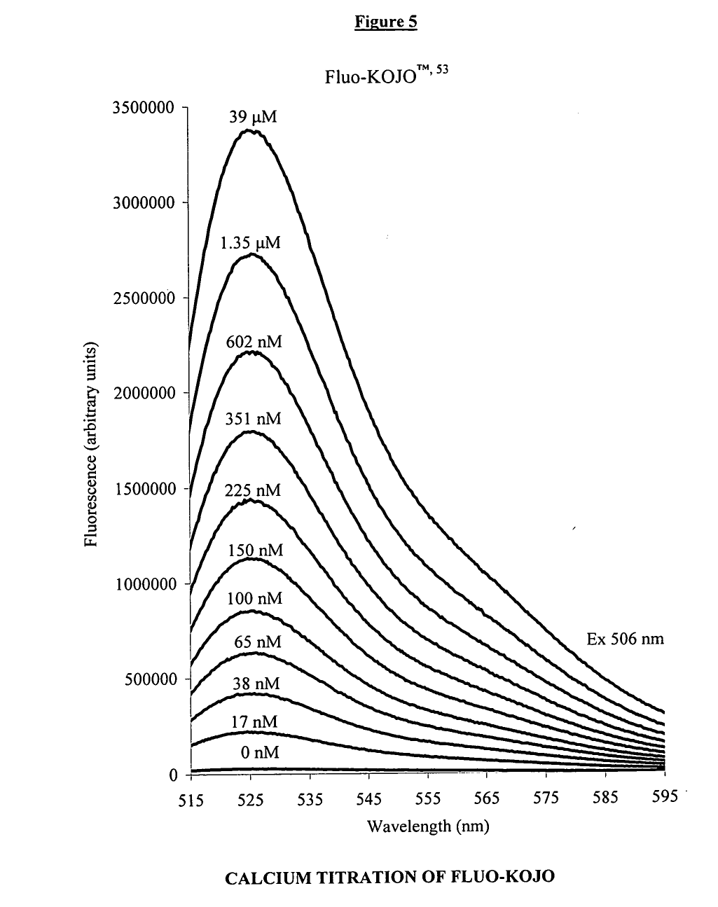

FIG. 5 shows a titration of FLUO-KOJO with calcium. It is an emission spectra of Ca²⁺ titration of FLUO-KOJO in 10 mM EGTA, 100 mM KCl, 30 mM MOPS buffer at pH 7.2. Excitation was set at 506 nm. The calcium concentrations added are sequentially as follows: 0 nM, 17 nM, 38 nM, 65 nM, 100 nM, 150 nM, 225 nM, 351 nM, 602 nM, 1.35 μM, and 39 μM. The temperature was set at 250 and that gave a dissociation constant of 419 nM.

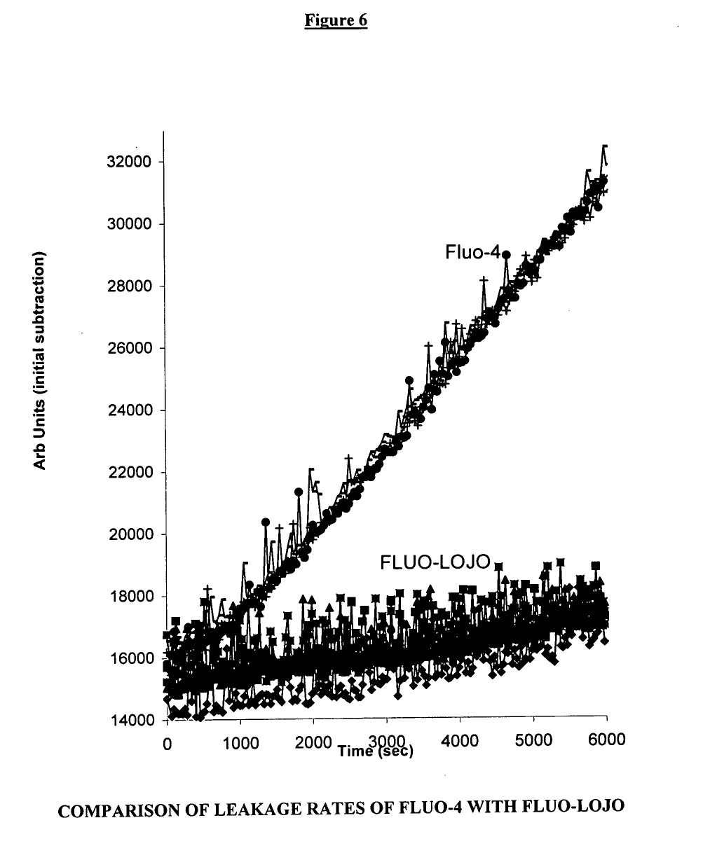

FIG. 6 shows the impact of dye leakage on apparent baseline or resting levels of cellular calcium. Rat cortical neurons were incubated for 20 mins with 2 μM solution FLUO-4(AM) at 20° C. The cells were washed for 10 mins to get rid of all external dye. 14 mM external calcium was added and the baseline was monitored on a BMG Galaxy fluorimeter. Excitation was 488 nm with emission collected at 515 nm to obtain the FLUO-4 trace. For the FLUO-LOJO trace, rat cortical neurons were incubated with similar concentration 2 μM FLUO-LOJO at 37° for 60 minutes (conditions designed for leakage). The cells were washed for 10 mins to get rid of any external dye and again 14 mM external calcium was added and the baseline was monitored on a BMG Galaxy fluorimeter. Excitation was set at 488 nm and emission collected at 515 nm to obtain the FLUO-LOJO trace.

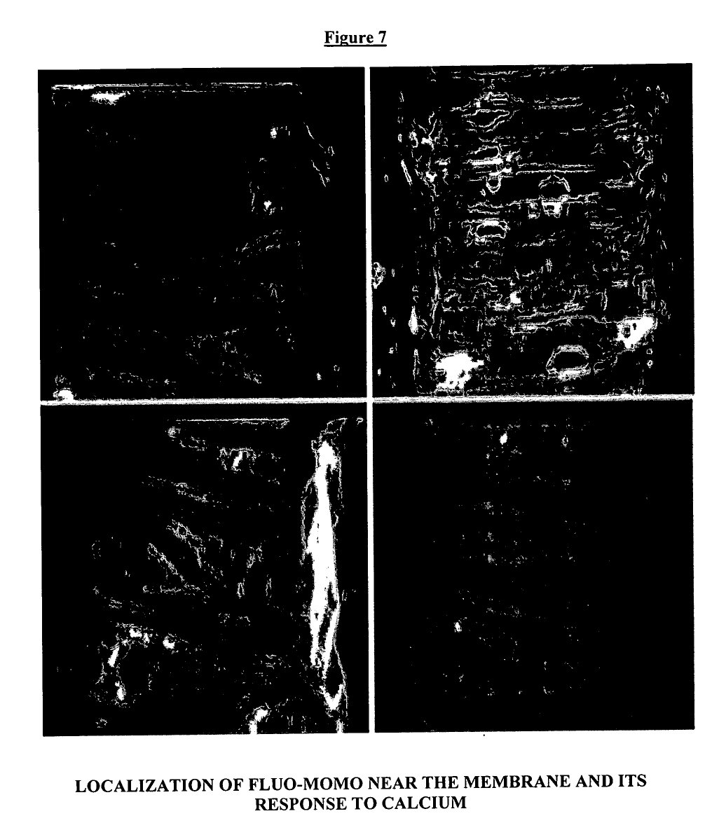

FIG. 7 shows localization of FLUO-MOMO and its response to calcium in the membrane of the walls of mouse small mesenteric arteries. The plane is tangential to the artery, very close to the coverslip. The spindle-shaped cells run horizontally, the artery lumen runs vertically. FLUO-MOMO (left, top and bottom) localizes to the plasma membrane. The FLUO-MOMO images are quite bright because the cells have been activated and [Ca²⁺] cyt has risen. The dye distribution is similar to that of FM4-64 (top right), which also localizes to the plasma membrane. In contrast, G-CaMP-2 (bottom right) is distributed uniformly in the cytosol of myocytes in a mouse small mesenteric artery. (Photo courtesy of Dr. Blaustein of The University of Maryland Medical School.)

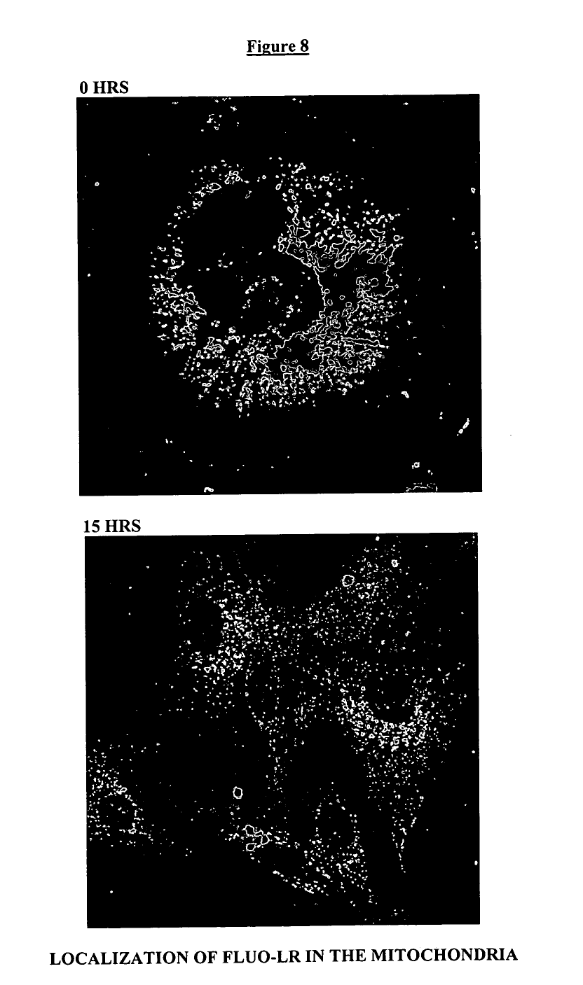

FIG. 8. Localization of FLUO-LR into mitochondria. Endothelial cells from normal and diabetic models were loaded with FLUO-LR (Cl)AM for 30 mins at 37° C. in cell growth media (i.e. buffer) lacking serum. Subsequently, cells were washed and further incubated for 30 mins at 37° C. Cell imaging was performed thereafter at t=o or t=15 hrs. In the case of t=15 hrs., cells were kept in the incubator in media (containing serum and growth factors, lacking dye). Imaging was performed using the Zeiss-LSM Meta 510 using 63× objective (water immersion, NA 1-4). Excitation was set at 488 (argon laser) for confocal experiments; or the Mira-Verdi (8W) mod-locked at 800 nm for multiphoton experiments.

### DETAILED DESCRIPTION OF THE INVENTION

The present invention consists of a new class of visible wavelength fluorescent calcium indicators with the BAPTA-like portion rendered zwitterionic by introduction of amine and carboxylic groups. “Fluo” compounds generally function with no extra ionized groups. The modifications include (a) piperazinoacetic acid for the leakage resistance (FLUO-LR XIII), (b) dodecylpiperazine for a near membrane indicator (FLUO-MOMO XIII, FLUO-NOMO XIII), and (c) a propionic acid for general leakage resistance (FLUO-LOJO XII, FLUO-KOJO XII).

The invention disclosed herein is a new family of visible wavelength calcium indicators designed as special derivatives of the BAPTA-like fluorescent calcium indicators FLUO-3 and FLUO-4, where modifications made to the right side BAPTA ring give a new functionality to the indicator while having a minimal impact on the fluorescent or chelation properties of the indicator. Different kinds of functionalities are obtained by changing the methyl of the BAPTA-like ring of “fluo” compounds to various R groups. When R is a straight chain propionic acid, a leakage resistant “fluo” compound is obtained by formation of a salt with the carboxylic acid in situ. If R contains a piperazine where one of the amines can be protonated in situ, two functions are obtained. When the free piperazine nitrogen is linked to a fatty dodecyl moiety, the “fluo” compound becomes hydrophobic enough to operate near the plasma membrane. When the free piperazine amine is attached to an acetic acid function, a zwitterion is created in situ, leading to leakage resistance again. Unlike UV indicators FuraPE3, though, this “fluo” compound tends to gravitate toward the mitochondria and never leaks out.

### DETAILED DESCRIPTION OF THE INVENTION—METHODS OF SYNTHESIS

Methods developed for synthesizing the new dyes relate to the synthesis of the “fluo” analogs shown below as structure A and structure B.

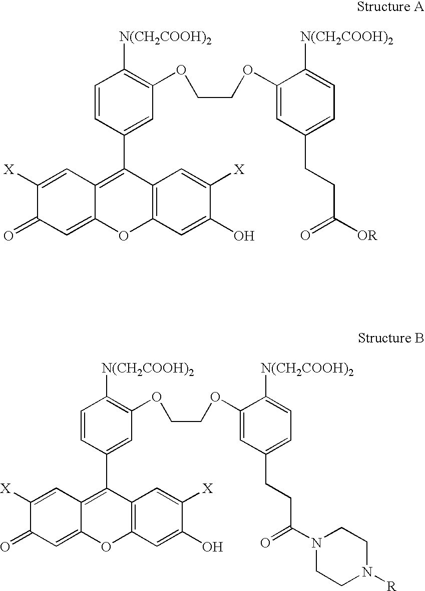

The present invention comprises a new class of visible wavelength fluorescent calcium-specific indicator dyes. Preferred examples that have been tested in cells and proven useful include FLUO-LOJO and FLUO-KOJO, FLUO-NOMO and FLUO-MOMO, and FLUO-LR. FIGS. 1 and 2 outline the synthetic route utilized in preparing these compounds. FIG. 3 outlines an alternate method for preparing FLUO-LOJO and FLUO-KOJO only. Full descriptions of the reaction conditions are described below. In all the figures and the discussion that follows, Roman numerals are used to identify the various chemical compounds.

The syntheses of the compounds claimed herein is described in the detailed synthesis of FLUO-LOJO, FLUO-KOJO, FLUO-NOMO, FLUO-MOMO, and FLUO-LR. Those skilled in the art will recognize new starting materials and new reactions. To that end, we point out the key intermediates VIII, XI, and XVI, and discuss their synthesis.

The key intermediate VIII shows a design of a “fluo” compound that does not use the usual 2-nitro-5-methylphenol, but a new starting material II, 3-hydro-4-nitrobenzaldehyde. The phosphorane IV chosen to react with the aldehyde III in a wittig reaction gives an ester with methyl function. This methyl function allows for a selective hydrolysis over a tert-butyl ester later in the synthesis. Compound VI, which has both the tert-butyl ester and methyl ester function was obtained by catalytic hydrogenation of V, followed by alkylation using tert-butylbromoacetate and ethyldiisopropylamine. The key intermediate VIII was then obtained by bromination of VI with bromine, followed by selective hydrolysis of the methyl ester using 3N potassium carbonate at 50° C. The next key intermediate XI in FIG. 2 had different synthesis depending on whether X=chloride for FLUO-3 analogs and X=fluoride for FLUO-4 analogs. When X was chloride, the starting material was commercial 3,3′,6,6′tetrahydroxybenzophenone. The benzophenone was chlorinated using sulfuryl chloride and ring closure effected by heating the chloride at 2200 in water. The final form of the intermediate XII where X=Cl was obtained by protecting the phenols with methoxymethyl chloride. When X=F in XII, the synthesis followed the procedure Chen et al in JACS, vol. 124, 3840 (2002)¹³. The piperazine derivative IX was obtained by reacting the bromide of the substituent with huge excess of pure piperazine and purifying by column chromatography. The coupling with the intermediate XII necessary to obtain the final products was done in all cases in THF at −100° C. using tert-butyllithium. The MEM derivatives of the final products were removed with acetic acid.

FIG. 3 is an alternate synthesis for FLUO-LOJO and FLUO-KOJO because it does not involve selective hydrolysis required for the other route. The coupling is also done using fluororesorcinol or chlororesorcinol in methanesulfonic acid and not the −100° C. reaction used in the regular synthesis. The critical intermediate XV is obtained by a vilsmeier reaction on XIV. The common intermediate V for both syntheses is modified by catalytic hydrogenation followed by alkylation with ethyl bromoacetate to give XIV, which has all base labile esters. The chlororesorcinol is obtained commercially, but the fluororesorcinol is obtained by electrophilic fluorination of resorcinol using selectfluor, see Banks et al in J.C.S. Chem. Comm., 595 (1992)¹⁴.

Compound Synthesis

Proton NMR spectra were recorded on a JEOL Eclipse⁺ instrument at 400 MHz. Peaks are reported below using the following convention: NMR, (solvent, operating frequency), chemical shift in ppm from tetramethylsilane, multiplicity (s=singlet, d=doublet, dd=doublet of doublets, t=triplet, q=quartet, m=multiplet, br=broad, spin-spin coupling constant if appropriate), integrated number of protons. In cases where adjacent peaks are too close for their integrals to be separated, the total integral for the cluster is given.

III Dinitroaldehyde C₁₅H₁₂N₂O₇ Mol. Wt.: 332.27

Compounds I (7.0 g, 28.4 mmol) and II (5.0 g, 32.4 mmol) were dissolved in DMF (15 mL) containing sodium carbonate (7 g) and heated at 140° C. for 1 hour. The mixture was allowed to cool to room temperature, then the salts were filtered and washed with DMF (15 mL). The filtrate was poured into water (300 mL) and the resulting precipitate was filtered, washed with water and dried to give III (7.38 g, 78% from I) as a tan powder. NMR, (CO(CD₃)₂) 10.131, s, 1H; 7.980, d, 1H; 7.946, d, 1H; 7.808, dd, 1H; 7.707, dd, 1H; 7.647, m, 1H; 7.431, dd, 1H; 7.154, m, 1H; 4.763, m, 4H.

V Wittig C₁₈H₁₆N₂O₈ Mol. Wt.: 388.33

The phosphorane salt of IV was prepared by adding methyl bromoacetate (11.75 mL, 124 mmol) to a solution of triphenylphosphine (30 g, 115 mmol) in benzene (100 mL), and stirring for 2 hours. The resulting precipitate was collected by vacuum filtration and dried under vacuum to give the bromide salt of IV (45.3 g) as a white solid. Subsequently, a mixture of III (13.0 g, 39.1 mmol), IV (24.3 g, 58.1 mmol), sodium carbonate (24.0 g) and DMF (40 mL) was stirred at 120° C. for 75 minutes. The resulting dark brown mixture was diluted with ethyl acetate (900 mL) and the salts were filtered. The filtrate was washed with brine 3 times, dried over sodium sulfate, and evaporated to dryness under vacuum. The resulting dark brown solid was triturated with methanol (20 mL) and the light tan solid was collected by filtration and dried well under vacuum to give V (12.69 g, 84% from III). NMR, (CO(CD₃)₂) 7.885-7.433, m, 7H; 7.154, m, 1H; 6.753, d, 1H; 4.687, m, 4H; 3.770, s, 3H.

VI Reduction/Alkylation C₄₂H₆₂N₂O₁₂ Mol. Wt.: 786.95

Compound V (13.7 g, 35.3 mmol) was dissolved in hot DMF (75 mL) and hydrogenated using 5% Pd/C (3.0 g) in a Parr Hydrogenator at 85 psi for 2.5 hours. The catalyst was removed by filtration and the solution was further hydrogenated using fresh 5% Pd/C (3.0 g) at 85 psi overnight to ensure the reduction of the stilbene. Afterwards, the catalyst was filtered and the filtrate was diluted with ethyl acetate (500 mL), washed with brine 3 times, dried over sodium sulfate and evaporated to dryness under vacuum to yield an off-white powder which was one spot by TLC. The solid intermediate was then dissolved in acetonitrile (45 mL) and diisopropylethylamine (35 mL, 200.9 mmol), to which tert-butyl bromoacetate (30 mL, 203.2) was added. The solution was stirred at 120° C. overnight, and upon completion, was diluted with 1L ethyl acetate, washed with pH 2 phosphate buffer twice and brine twice, then dried over sodium sulfate and evaporated to a viscous red gum under vacuum. The gum was triturated with methanol to yield a white solid which was collected by filtration and dried under vacuum to give VI (18.47 g, 72% from V). NMR, (CDCl₃) 6.929-6.844, m, 4H; 6.788-6.697, m, 3H; 4.374-4.313, m, 4H; 4.064, s, 4H; 4.033, s, 4H; 3.652,s, 3H; 2.824, t, 2H; 2.665, t, 2H; 1.411, s, 18H; 1.404, s, 18H.

VII Bromination C₄₂H₆₁BrN₂O₁₂ Mol. Wt.: 865.84

Compound VI (21.37 g, 27.2 mmol) was dissolved in dichloromethane (60 mL) and pyridine (3 mL) was added. Bromine (1.68 mL, 32.8 mmol) was dissolved in dichloromethane (23 mL). Both solutions were chilled in dry ice/isopropanol baths, and the latter was added to the former dropwise with stirring. Upon complete addition the solution was diluted with dichloromethane (100 mL), washed with water, 2% sodium thiosulfate solution, twice with brine, dried over sodium sulfate and filtered. The filtrate was evaporated under vacuum to yield an orange gum, which was triturated with hexane, filtered, and dried to give VII (18.8 g, 80% from VI) as white powder. NMR, (CDCl₃) 7.036-6.983, m, 2H; 6.806-6.711, m, 4H; 4.330, q, 4H; 4.021, s, 8H; 3.654, s, 3H; 2.850, t, 2H; 2.569, t, 2H; 1.409, s, 36H.

VIII Selective Methyl Ester Hydrolysis C₄₁H₅₉BrN₂O₁₂ Mol. Wt.: 851.82

To a stirred solution of VII (10.4 g, 12.0 mmol) in methanol (125 mL) and dioxane (75 mL) was added 3M potassium carbonate (35 mL). The solution was heated to 50° C. and monitored by TLC to minimize the hydrolysis of more than 1 ester. After 1.5 hours the solution was poured over ethyl acetate (800 mL) and the stirred solution was neutralized with acetic acid (16 mL). The solution was washed with brine twice, dried over sodium sulfate, and evaporated to an orange gum under vacuum. Purification by silica gel chromatography, eluting with 20-50% ethyl acetate in hexane gave recovered VII (1.26 g) and VIII (7.94 g, 78% yield, 90% recovery) as a light yellow foam. NMR, (CDCl₃) 7.022-6.959, m, 3H; 6.803-6.717, m, 4H; 4.307, t, 4H; 4.021, s, 8H; 2.868, t, 2H; 2.614, t, 2H; 1.410, s, 36H.

IXa LR Piperazine C₁₀H₂₀N₂O₂ Mol. Wt.: 200.28

A solution of tert-butylbromoacetate (20.67 mL, 140 mmol) in chloroform (300 mL) was added dropwise to a stirred solution of piperazine (50 g, 580 mmol) in chloroform (300 mL) at room temperature over 2 hours. The resulting salts were filtered and washed with chloroform, and the filtrate was diluted to 1 L with chloroform and washed with water, which was back extracted with chloroform twice. The combined organics were dried over sodium sulfate, concentrated under vacuum, and purified by silica gel chromatography, eluting with 0-5% methanol in chloroform to furnish IXa. NMR, (CDCl₃) 3.100, s, 2H; 2.937, t, 4H; 2.545, t, 4H; 1.737, s, 1H; 1.467, s, 9H.

IXb NM Piperazine C₁₆H₃₄N₂ Mol. Wt.: 254.45

A solution of 56 mL (233 mmol) 1-bromododecane in 200 mL chloroform was added dropwise to a stirred solution of 80 g (930 mmol) piperazine in 600 mL chloroform at room temperature over 5 hours. The resulting salts were filtered and washed with chloroform, and the filtrate was diluted to 1.6L with chloroform and washed with water, which was back extracted with chloroform twice. The combined organics were dried over sodium sulfate, concentrated under vacuum, and purified by silica gel chromatography, eluting with 2-15% methanol in chloroform to furnish IXb as a light yellow solid which melts readily. NMR, (CDCl₃) 4.278, s, 2H; 3.026, t, 4H; 2.549, s, br, 4H; 2.350, q, 2H; 1.480, t, 2H; 1.272, d, 18H; 0.881, t, 3H.

Xa LR Bromide C₅₁H₇₇BrN₄O₁₃ Mol. Wt.: 1034.08

Compound VIII (3.00 g, 3.5 mmol), disuccinimidyl carbonate (2.71 g, 10.6 mmol) and 4-(dimethylamino)pyridine (g, 10.6 mmol) were dissolved in anhydrous dimethylformamide (36 mL) and stirred at room temperature for 2 hours. Compound IXa (2.12 g, 10.6 mmol) was dissolved in anhydrous dichloromethane (8 mL), added to the reaction solution, and stirred overnight. The solution was diluted with 600 mL ethyl acetate, washed with pH 2 phosphate buffer and brine twice, dried over sodium sulfate and filtered. The filtrate was evaporated to dryness under vacuum to yield a yellow gum which was purified by silica gel chromatography, eluting with 50-70% ethyl acetate in hexane, to give Xa (3.40 g, 94% from VIII) as a light yellow semisolid. NMR, (CDCl₃) 7.006, m, 2H; 6.752, m, 4H; 4.332, m, 4H; 4.022, s, 8H; 3.670, t, 2H; 3.455, t, 2H; 3.124, s, 2H; 2.836, t, 2H; 2.546, m, 6H; 1.461, s, 9H; 1.414, s, 36H.

Xb NM Bromide C₅₇H₉₁BrN₄O₁₁ Mol. Wt.: 1088.26

Compound VIII (4.63 g, 5.4 mmol), disuccinimidyl carbonate (4.18 g, 16.3 mmol) and 4-(dimethylamino)pyridine (1.99 g, 16.3 mmol) were dissolved in anhydrous dimethylformamide (60 mL) and stirred at room temperature for 2 hours. Compound XIb (4.14 g, 16.3 mmol) was dissolved in anhydrous dichloromethane (30 mL), added to the reaction solution, and stirred overnight. The solution was diluted with 800 mL ethyl acetate, washed with pH 2 phosphate buffer and brine twice, dried over sodium sulfate and filtered. The filtrate was evaporated to dryness under vacuum to yield a yellow gum which was purified by silica gel chromatography, eluting with 50-70% ethyl acetate in hexane, to give Xb (4.95 g, 84% from VIII) as a yellow semisolid. NMR, (CDCl₃) 7.005, m, 2H; 6.754, m, 4H; 4.335, m, 4H; 4.022, s, 8H; 3.630, t, 2H; 3.412, t, 2H; 2.836, t, 2H; 2.553, t, 2H; 2.359, m, 6H; 1.460, br, 2H; 1.414, s, 36H; 1.256, s, 18H; 0.878, t, 3H.

XIa MEM Chloro Xanthone C₂₁H₂₂Cl₂O₈ Mol. Wt.: 473.30

Compound XIa was obtained by di-chlorinating benzophenone with sulfuryl chloride, then heating 3,3′-dichlorobenzophenone with water in a sealed vessel to furnish 2,7-dichloro-3,6-dihydroxyxanthone, which was subsequently treated with sodium hydride and methoxyethoxymethyl chloride. NMR (CDCl₃) 8.289, s, 2H; 7.272, s, 2H; 5.479, s, 4H; 3.908, m, 4H; 3.592, m, 4H; 3.389, s, 6H.

XIb MEM Fluoro Xanthone C₂₁H₂₂F₂O₈ Mol. Wt.: 440.39

Compound XIb was obtained by the method of Chen et al, with some slight modifications, from commercially available 2,4,5-trifluorobenzonitrile and resorcinol. Methoxylation of 2,4,5-trifluorobenzonitrile with sodium methoxide was followed by nitrile hydrolysis by potassium hydroxide, then treatment with thionyl chloride to furnish 5-fluoro-2,4-dimethoxybenzoyl chloride. Resorcinol was fluorinated with Selectfluor, then methoxylated with dimethyl sulfate to give 2,4-dimethoxyfluorobenzene. A one step acylation/demethylation was achieved with 5-fluoro-2,4-dimethoxybenzoyl chloride, 2,4-dimethoxyfluorobenzene and aluminum chloride in nitrobenzene to yield 5,5′-difluoro-2,2′,4,4′-tetrahydroxybenzophenone. 5,5′-difluoro-2,2′,4,4′-tetrahydroxybenzophenone was heated in a sealed vessel with water to afford 2,7-difluoro-3,6-dihydroxyxanthone, which was subsequently treated with sodium hydride and methoxyethoxymethyl chloride to give XIb. NMR, (CDCl₃) 7.949, d, 2H; 7.304, d, 2H; 5.456, s, 4H; 3.908, m, 4H; 3.593, m, 4H; 3.390, s, 6H.

XIIa KOJO

Aldehyde (6.000 g, 8.55 mmol) and 4-chlororesorcinol (3.702 g, 25.62 mmol) are combined and melted together at 140° C. To the molten gum is added methanesulfonic acid (18 mL) and the solution is stirred at 140° C. for 45 minutes. The reaction is cooled briefly, then quenched over ice, washing the tarry material out with methanol. The reaction is repeated at equivalent scale and the crude materials are combined.

The esters are hydrolyzed by adding ION KOH (65 mL, plus 5-10 mL more if needed to achieve a pH>10) to the cold mixture and stirring for 3 or more hours at r.t., removing the solvent in vacuo, redissolving in minimal water, and stirring one hour more at r.t. Acidification with HCl precipitates the crude penta-carboxylic acid, which is filtered and dried well in vacuo with P₂O₅.

In order to purify more easily, the crude penta-carboxylic acid (17.460 g) is benzylated with benzyl bromide (48 mL, 404 mmol) and diisopropylethylamine (80 mL, 460 mmol) in DMF (100 mL) at room temperature, overnight. The reaction is diluted with ethyl acetate (500 mL), washed with brine three times, dried over sodium sulfate and filtered. The filtrate is concentrated in vacuo and purified by flash chromatography (silica gel, 30-60% ethyl acetate in hexane eluent) to furnish 3.57 g benzylated material.

Cleavage of the benzyl esters is achieved by stirring the material in trifluoroacetic acid (188 mL) and thioanisole (88 mL) at room temperature for 2 days. The trifluoroacetic acid is removed in vacuo, and the reaction is quenched with ice chips. The solution pH is adjusted to 12-13 by the addition of ION KOH. The upper layer of thioanisole is separated and discarded, and residual thioanisole is removed in part by extraction with ethyl acetate and chloroform. To the vigorously stirred aqueous solution is added conc. HCl to precipitate the relatively pure penta-carboxylic acid (2.16 g), from which the biologically useful hexa-K⁺ and hexa-AM ester derivatives can by prepared.

XIIb LOJO

Aldehyde (6.000 g, 8.55 mmol) and 4-fluororesorcinol (3.282 g, 25.64 mmol) are combined and melted together at 120° C. To the molten gum is added methanesulfonic acid (18 mL) and the solution is stirred at 120° C. for 85 minutes. The reaction is cooled briefly, then quenched over ice, washing the tarry material out with methanol.

The esters are hydrolyzed by adding ION KOH (30 mL, plus 5-10 mL more if needed to achieve a pH>10) to the cold mixture and stirring for 3 or more hours at r.t., removing the solvent in vacuo, redissolving in minimal water, and stirring one hour more at r.t. Acidification with HCl precipitates the crude penta-carboxylic acid, which is filtered and dried well in vacuo with P₂O₅.

In order to purify more easily, the crude penta-carboxylic acid (7.036 g) is benzylated with benzyl bromide (18.5 mL, 156 mmol) and diisopropylethylamine (32 mL, 184 mmol) in DMF (40 mL) at room temperature, overnight.

The reaction is diluted with ethyl acetate (500 mL), washed with brine three times, dried over sodium sulfate and filtered. The filtrate is concentrated in vacuo and purified by flash chromatography (silica gel, 30-60% ethyl acetate in hexane eluent) to furnish 1.578 g benzylated material.

Cleavage of the benzyl esters is achieved by stirring the material in trifluoroacetic acid (80 mL) and thioanisole (40 mL) at 0° C. initially, followed by r.t. for 2 days. The trifluoroacetic acid is removed in vacuo, and the reaction is quenched with ice chips. The solution pH is adjusted to 12-13 by the addition of ION KOH. The upper layer of thioanisole is separated and discarded, and residual thioanisole is removed in part by extraction with ethyl acetate and chloroform. To the vigorously stirred aqueous solution is added conc. HCl to precipitate the relatively pure penta-carboxylic acid (563mg), from which the biologically useful hexa-K⁺ and hexa-AM ester derivatives can be prepared.

XIII

X (1 mmol) was placed in a 100 mL round bottom flask and kept under argon via a Claisen adaptor. Tetrahydrofuran (THF, 15 mL) was added to dissolve X and the solution was diluted with methyltetrahydrofuran (15 mL). The solution was kept at −100° C. (ether/liquid nitrogen) for 10 minutes. Tertiary-butyl lithium (6 equivalents for 4 ester groups and 7 equivalents for 5 ester groups) was added slowly to maintain the −100° C. temperature. The lithiated compound was allowed to equilibrate for 1 hour and XI (1.2 mmol) in THF (10 mL) was added at −100° C. The reaction mixture was allowed to warm up slowly to −20° C. over 1 hour, then diluted with ethyl acetate and washed with brine. The residue after drying and evaporating off the organic solvent was purified by silica gel chromatography.

The coupled dye was treated with excess trifluoroacetic acid overnight. Water and ice were added and the pH adjusted to 2 using ION KOH. Solid precipitates were formed and filtered. For titration purposes the free acid was purified by preparative silica gel thin layer chromatography using the solvent ratio 10 chloroform: 5 methanol: 1 water: 0.1 acetic acid. The products were extracted with 10 chloroform: 10 methanol: 1 water: 0.1 acetic acid. The solvents were evaporated in vacuo after extraction and potassium salts were made for an aqueous titration. For cell loading purposes, acetoxymethyl derivatives were formed directly from the free acid without preparative thin layer chromatography purification. The free acids were dissolved in dry DMF, and diisopropylethylamine and acetoxymethyl bromide were added. The reaction was monitored to completion, and the products were purified by silica gel chromatography.

XIV Reduction/Alkylation C₃₄H₄₆N₂O₁₂ Mol. Wt.: 674.74

Compound V (18.0 g, 46.4 mmol) was dissolved in hot DMF (100 mL) and hydrogenated using 5% Pd/C (2.5 g) in a Parr Hydrogenator at 80 psi for 2.5 hours. The catalyst was removed by filtration and the solution was further hydrogenated using fresh 5% Pd/C (2.5 g ) at 80 psi overnight to ensure the reduction of the stilbene. Afterwards, the catalyst was filtered and the filtrate was diluted with ethyl acetate (400 mL), washed with brine 3 times, dried over sodium sulfate and evaporated to dryness under vacuum to yield an off-white powder which was one spot by TLC. The solid intermediate was then dissolved in acetonitrile (60 mL) and diisopropylethylamine (48 mL, 200.9 mmol), to which ethyl bromoacetate (31 mL, 203.2 mmol) was added. The solution was stirred at 120° C. overnight, and upon completion, was diluted with ethyl acetate (500 mL), washed with pH 2 phosphate buffer twice and brine twice, then dried over sodium sulfate and evaporated to a viscous red gum under vacuum. The gum was triturated with methanol to yield a white solid which was collected by filtration and dried under vacuum to give VI (22.50 g, 72% from V). NMR, (CDCl₃) 6.845-6.927, m, 4H; 6.695-6.798, m, 3H; 4.310-4.374, m, 4H; 4.064, s, 4H; 4.030, s, 4H; 4.011-4.076, m, 8H; 3.656,s, 3H; 2.787-2.864, t, 2H; 2.546-2.645, t, 2H; 1.116-1.158, dt, 12H.

XV Vilsmeier Formylation C₃₆H₄₇N₂O₁₃ Mol. Wt.: 715.76

Compound VI (22.50 g, 33.38 mmol) was dissolved in DMF (60 mL) with pyridine (25 mL), and stirred at 0° C. for twenty minutes. Phosphorus oxychloride (25 mL, 268 mmol) was added to the solution dropwise with stirring over twenty minutes. Upon complete addition the solution was heated at 60° C. overnight. The reaction was quenched over ice and dissolved with ethyl acetate and water. The aqueous layer was separated and extracted three times with ethyl acetate. The combined organics were washed with brine twice, dried over sodium sulfate and filtered. The filtrate was evaporated under vacuum to yield a dirty yellow solid which was triturated with methanol, filtered, and dried to give XV (13.87 g, 58% from XIV) as an off-white powder. NMR, (CDCl₃) 9.798, s, 1H; 7.373-7.393, m, 2H; 6.686-6.769, m, 4H; 4.251-4.318, m, 4H; 4.228, s, 4H; 4.118, s, 4H; 4.011-4.072,m, 8H; 3.672, s, 3H; 2.841-2.861, t, 2H; 2.549-2.588, t, 2H; 1.118-1.157, dt, 12H.

XVIA

This is a commercially available chloro version of Resorcinol.

XVIb

4-Fluororesorcinol A solution of SELECTFLUOR (200 g, 564.6 mmol) in acetonitrile (2.4L) and water (600 mL) is added dropwise to a stirred solution of resorcinol (41.52 g, 377 mmol) in acetonitrile (750 mL), at room temperature. Upon completion of addition the solvent is removed in vacuo, and the residue is redissolved in ethyl acetate. The salts are filtered and the filtrate is concentrated in vacuo. The residue is purified by silica gel chromatography, eluting with 17-25% diethyl ether in hexane, to furnish XVIb (30 g, 62% yield from resorcinol).

Those of skill in the art will recognize various changes and modifications which may be made to the examples described above. These changes and modifications are intended to be within the scope of the following claims.

### REFERENCES

The present specification refers to the following patents and publications, each of which is expressly incorporated by reference herein.
1. Tsien, R. Y., Biochemistry 19:2396 (1980)
2. Grynkiewicz, G., Poenie, M., and Tsien, R. Y., J. Biol. Chem. 260:3440 (1985)
3. Vorndran, C., Minta, A., and Poenie, M., M. Biophysical J. 69:2121 (1995)
4. Minta, A., and Tsien, R. Y., J. Biol. Chem. 264:8171 (1989)
5. Gee, K. R., Brown, K. A., Chen, W-N V., Bishop-Stewart, J., Gray, D., and Johnson, I., Cell Calcium 27:97 (2000)
6. Thomas, D., Tovey, S. C., Collins, T. J., Bootman, M. D., Berridge, M. J., and Lipp, P., Cell Calcium 28:213 (2000)
7. Tsien, R. Y., Nature p. 527 (1980)
8. Chen, C. A., Yeh, R. H., and Lawrence, D. S., JACS 124:3840
9. Banks, E. R., Mohialdin-Khaffat, S. N., Lal, G. S., Sharif, I., and Syvret, R. G. J. Chem. Soc. Chem. Comm. 599 (1992)

1. A class of cellular visible wavelength calcium indicators which are leakage resistant and consisting of a chemical compound having the formula 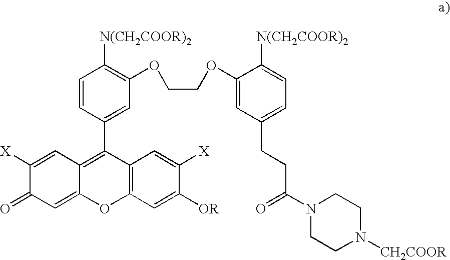
Where R=K⁺ or CH₂OCOCH₃ (acetoxymethyl ester)
X=Cl, F
or 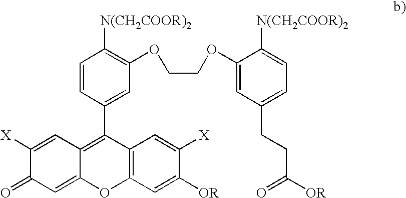
Where R =K⁺ or CH₂OCOCH₃ (acetoxymethyl ester)
X=Cl,F

2. A class of visible wavelength calcium indicators which operate near membranes and consisting of a chemical compound having the formula 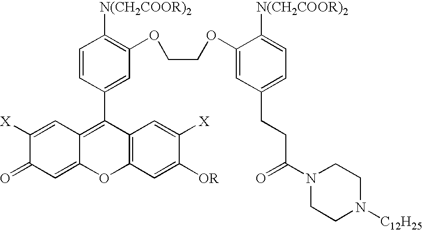
Where R =K⁺ or CH₂OCOCH₃ (acetoxymethyl ester)
X=Cl, F
